(19) United States
(12) Patent Application Publication
(10) Pub. No.: US 2021/0267530 A1
(43) Pub. Date: Sep. 2, 2021

(54) MULTICLASS CLASSIFICATION METHOD FOR THE ESTIMATION OF EEG SIGNAL QUALITY

(71) Applicant: MYBRAIN TECHNOLOGIES, Issy-les-Moulineaux (FR)

(72) Inventors: Fanny GROSSELIN, Paris (FR); Xavier NAVARRO-SUNE, Paris (FR); Yohan ATTAL, Paris (FR)

(73) Assignee: MYBRAIN TECHNOLOGIES, Issy-les-Moulineaux (FR)

(21) Appl. No.: 17/251,919

(22) PCT Filed: Jun. 27, 2019
(86) PCT No.: PCT/EP2019/067191

§ 371 Date: Dec. 14, 2020

(30) Foreign Application Priority Data
Jun. 29, 2018 (EP) 18305841.1

(51) Int. Cl. A61B 5/372; A61B 5/00; G16H 50/20; G16H 10/00

(52) U.S. Cl. CPC A61B 5/372; A61B 5/7207; A61B 5/7267; A61B 5/7257; A61B 2562/04; A61B 5/7221; G16H 50/20; G16H 10/00; A61B 5/6843; A61B 5/725

(57) ABSTRACT

A method for assessing an electroencephalographic signal quality based on a multiclass classification, including: receiving at least one segment of electroencephalographic signal from at least one electrode; extracting at least one feature value from each electroencephalographic signal segment channel; classifying with a first classification to assign each electroencephalographic signal segment channel to one of at least three quality classes. The first classification is performed by a k-nearest neighbors' algorithm: using a first training set of multiple training samples, each training sample being associated to a quality class and to at least one feature value; and assigning to each electroencephalographic signal segment channel the quality class which is the most frequent class among the training samples of the first training set nearer to each electroencephalographic signal segment channel; the distance is calculated between the feature value of each electroencephalographic signal segment channel and each feature value of the training samples.

### FIELD OF INVENTION

The present invention pertains to the field of signal processing. In particular, the invention relates to a method to assess the electroencephalographic signal quality based on multiclass classification method.

### BACKGROUND OF INVENTION

The electroencephalography (EEG) is an electrophysiological monitoring method to record electrical activity of the brain. It is typically noninvasive, with the electrodes placed along the scalp. EEG is widely used in the diagnosis of brain related diseases like sleep disorders, epilepsy, neurological disorders, etc. It may also be used to allow control of a Brain-computer interface (BCI), a device which allows direct control of a computer or device via the modulation of electrical activity in the brain. However, the electroencephalographic signals are almost always contaminated by the overlapping with other electrical signals not generated by the brain activity, the so-called artifacts.

Electroencephalographic signals may be contaminated by environmental or biological artifacts. Environmental artifacts are non-physiological artifacts due to device and recording equipment like interference from electric fields, poor electrode connection, electro-magnetic interferences by electronic devices in the environment near to EEG amplifiers, alternative current at either 50 or 60 Hz, cable movement, sweating etc. The environmental artifacts are often referred to as noise. Biological artifacts are mainly caused by movements of the subject like eye or head movements, muscle contraction and cardiac artifacts can affect as well EEG.

The amplitude of artifacts can be quite large relative to the size of amplitude of the cortical signals of interest. Therefore, effective detection of artifacts directly impacts the interpretation of electroencephalographic signals clinically.

Multiple strategies are actually known to reduce the intensity or avoid these undesired disturbances like registering into electromagnetic shield room to avoid external electro-magnetic perturbations. Skin preparation for conductive gel electrode can be used to reduce the impedance between the sensors and the skin. In research laboratories, it is also possible to ask to the subjects to avoid facial movement in order to reduce the muscular artifacts. But these solutions are not always implementable, especially in hospital or for wearable EEG systems with wet or dry sensors. Thus, the assessment of the EEG quality is particularly important in these cases where the EEG environment is not controlled.

Several methods are already proposed to check the quality of EEG signals. The measure of skin-sensor contact impedance is commonly used. It is generally assumed that a low impedance (lower than 5 kΩ) is associated to a good contact and therefore a good quality for the EEG signal. However, a good contact does not mean that the EEG signal is artifacts free. Moreover, impedance measurements to assess the quality of the skin-electrodes contact will raise the cost of wearable EEG systems.

In brain-computer interfaces, it is possible to assess EEG signal quality by doing some performance measures on the sensors in several applications: signal-to-noise ratios of steady-state visually evoked potentials, change in P300 components of event related potentials and the like.

The quality of EEG signal can be also assessed by the use of measures on the amplitude distribution of the signal which is supposed to be Gaussian for good quality EEG signals. These statistic measures include probability distribution, mean, standard deviation, skewness and kurtosis. For instance, amplifier drifts or equipment artifacts are detected by shifts in values of the mean signal amplitude while artifact generated by strong muscle activity are detected by the kurtosis since they are characterized by a very peaky signal.

Artifact detection based on thresholding is a simple, widely used method that removes EEG segments exceeding a value (threshold) based on the above mentioned descriptive statistics. The problem with using threshold-based approaches is that the boundary between a good quality and a bad quality EEG signal is fixed by one or more predefined values and, contrary to classifier-based methods, there is no control over the compromise between the true positive/negative rates and the false detections rate. Indeed, a classifier uses elements of pattern recognition to find a probabilistic decision rule for classifying a set of features. This type of probabilistic method allows to adapt the decision rule to the desired performance of classification problem.

### SUMMARY

A first aspect of the present invention relates to a method for assessing the quality of an electroencephalographic signal (EEG) based on a multiclass classification, wherein said method comprises the following steps:
receiving at least one segment of electroencephalographic signal acquired from at least one electrode;
extracting at least one feature value from the electroencephalographic signal segment;
classifying with a first classification so to assign the electroencephalographic signal segment to one of at least three quality classes: {TAG1, TAG2, . . . , TAGN};
   wherein said first classification is performed by a k-nearest neighbors' algorithm:
using a first training set comprising multiples training samples, wherein each training sample of the first training set is associated to one of the quality classes and to at least one feature value; and
assigning to the electroencephalographic signal segment the quality class which is the most frequent class among the k training samples of the first training set which are nearer to said the electroencephalographic signal segment; wherein the distance is calculated between the feature value of electroencephalographic signal segment and each feature value of the training samples.

According to one embodiment, the method for assessing the quality of an electroencephalographic signal based on a multiclass classification, wherein said method comprises the following steps:
receiving at least one segment of electroencephalographic signal acquired from at least two electrodes;
extracting at least one feature value from each channel of the electroencephalographic signal segment;
classifying with a first classification so as to assign each channel of the electroencephalographic signal segment to one of at least three quality classes (TAG): {TAG₁, TAG₂, . . . , TAG_{N}};
wherein said first classification is performed by a k-nearest neighbors' algorithm:
using a first training set comprising multiples training samples, wherein each training sample of the first training set is associated to one of the quality classes and to at least one feature value; and
assigning to each channel of the electroencephalographic signal segment the quality class which is the most frequent class among the k training samples of the first training set which are nearer to each channel of the electroencephalographic signal segment; wherein the distance is calculated between the feature value of each channel of the electroencephalographic signal segment and each feature value of the training samples.

The method of the present invention analyses the segment of electroencephalographic signal channel by channel which advantageously allow to provide in real time an accurate information for each channel about the quality of the collected EEG signal for an immediate feedback to a user of a portable EEG system, so as to improve the positioning of the electrodes of EEG system if necessary.

Furthermore, the present method is constructed to allow the analysis of segment of electroencephalographic signal received from more than two EEG channels.

According to one embodiment, the segment of electroencephalographic signal (S) is acquired from at least two electrodes.

According to one embodiment, the at least one feature and the k value of the k-nearest neighbors' algorithm are configured so that:
the first quality class is associated to EEG signal segment acquired with the electrodes positioned according to a first predefined configuration of contact between the electrodes and a subject' scalp and during a first predefined physiological state of a subject; and/or
the second quality class is associated to EEG signal segments acquired with the electrodes positioned according to a first predefined configuration of contact between the electrodes and a subject' scalp and during a second physiological state of a subject; and/or
the third quality class corresponds to EEG signal segments acquired with the electrodes positioned according to a second predefined configuration of contact between the electrodes and a subject' scalp.

According to one embodiment, the feature is a quality index function of the standard deviation of the electroencephalographic signal segment.

According to one embodiment, the quality index is further function of kurtosis, maximum of absolute value and/or median of absolute values

According to one embodiment, the at least one feature of the electroencephalographic signal segment is chosen from the following list of features:
the rate of zero-crossings of the electroencephalographic signal segment over a fixed threshold;
power spectrum moments of different orders;
index of spectral deformation;
modified median frequency.

According to one embodiment, the first classification is performed by a weighted k-nearest neighbors' algorithm.

According to one embodiment, the method further comprises a second classification assigning the electroencephalographic signal segment classified in quality class to one of at least two non-exploitable classes: {TAG_N1, TAG_N2, . . . TAG_NN}, wherein the electroencephalographic signal segment classified in the non-exploitable classes (TAG_N) are the electroencephalographic signal segments excluded from further analysis.

According to one embodiment, the second classification is performed with a weighted k-nearest neighbors' algorithm using a second training set, said second training set comprising multiples training samples, wherein each training sample of the second training set is associated to one of the non-exploitable classes and to at least one feature value.

According to one embodiment, the at least one feature and the k value of the k-nearest neighbors' algorithm are configured so that:
the first non-exploitable class is associated to EEG signal segment acquired with the electrodes positioned according a second predefined configuration of contact between the electrodes and a subject' scalp; and
the second non-exploitable class is associated to EEG signal segment acquired with electrodes having no physical contact with a subject's scalp.

According to one embodiment, the method further comprises a step for the discrimination of muscular artifacts from other source artifacts in electroencephalographic signal (EEG), said method comprising:
for each EEG signal segment classified in the quality class (TAG₂) computing the spectrum by Fourier transform in a predefined frequency range;
calculating of a spectral distance between the spectrum of each EEG signal segment and a reference spectrum; and
comparing said spectral distance to a predefined threshold to determine the presence of a muscular artifact in the EEG signal segment in the quality class (TAG2) and assign it to a class (TAG_{2_}m).

According to one embodiment, the reference spectrum is computed as the average value of the spectra of at least two electroencephalographic signal segments, wherein said at least two electroencephalographic signal segments are acquired with the electrodes positioned according to a first predefined configuration of contact between the electrodes and a subject' scalp and during a first predefined physiological state of a subject.

According to one embodiment, the spectral distance is an Itakura spectral distance.

A second aspect of the present invention relates to a method for identifying muscular artifacts from other source artifacts in electroencephalographic signal (EEG), said method comprising:
receiving at least one electroencephalographic signal segment which comprises at least a signal contribution arising from one artifact source from a predefined list of artifact sources;
computing a spectrum of said EEG signal segment by Fourier transform in a frequency range;
calculating a spectral distance between the spectrum and a reference spectrum; and
comparing said spectral distance to a predefined threshold to determine the presence of a muscular artifact in the EEG signal segment.

According to one embodiment, the reference spectrum is computed as the average value of the spectra of at least two electroencephalographic signal segments, wherein said at least two electroencephalographic signal segments are acquired with the electrodes positioned according to a first predefined configuration of contact between the electrodes and a subject' scalp and during a first predefined physiological state of a subject

According to one embodiment, the spectral distance is an Itakura spectral distance.

The present invention further relates to a method for multiclass classification of an electroencephalographic signal (EEG), comprising the steps of
identifying an artifact in at least one electroencephalographic signal segment with the method according to any one of embodiments described hereabove;
among the electroencephalographic signal segment identified as comprising artifacts, identifying an electroencephalographic signal segment comprising muscular artifacts with the method according to any one of embodiments described hereabove.

Yet another aspect of the present invention concerns a method for updating a database, said method comprising steps of:
receiving a first set of pseudonymized data concerning a first subject; wherein said first set of pseudonymized data comprises at least one segment of electroencephalographic signal segment and a class to which said segment of electroencephalographic signal has been previously associated by the method according to any one of the embodiments described hereabove; and
updating said first database by storing the first set of pseudonymized data concerning the first subject.

The present invention further relates to a system comprising a data processing system comprising means for carrying out the steps of the method according to any one of the embodiments described hereabove.

According to one embodiment, the system comprises an acquisition set-up for acquiring at least a segment of electroencephalographic signals from a subject

The present invention further relates to a computer program product for multiclass classification of an electroencephalographic signal, the computer program product comprising instructions which, when the program is executed by a computer, cause the computer to carry out the steps of the method according to any one of the embodiments described hereabove.

The present invention further relates to a computer-readable storage medium comprising instructions which, when the program is executed by a computer, cause the computer to carry out the steps of the method according to any one of the embodiments described hereabove.

### Definitions

In the present invention, the following terms have the following meanings:
As used herein the singular forms “a”, “an”, and “the” include plural reference unless the context clearly dictates otherwise.
“Electroencephalogram” or “EEG” refers to the tracing of brain waves, by recording the electrical activity of the brain from the scalp, made by an electroencephalograph.
“Electroencephalograph” refers to an apparatus for amplifying and recording brain waves.
“Epoch” refers to a determined period or slice of neural signals.
“Single-channel” refers to an EEG signal composed by a one-dimensional signal.
“Feature” refers to an individual measurable property or characteristic of a signal being observed.
“Frequency band” refers to a specific range of frequencies in the spectrum of electroencephalographic signals.
“Real time” refers to a process for which the output is given within a time delay that is considered as smaller than the time delay required to perform the underlying task of modulation adequately. Therefore, for self-paced modulation, real time refers to a process implemented in less than 2000 ms, preferably less than 1500 ms, more preferably less than 1000 ms.
“Subject” refers to a mammal, preferably a human. In the sense of the present invention, a subject may be an individual having any mental or physical disorder requiring regular or frequent medication or may be a patient, i.e. a person receiving medical attention, undergoing or having underwent a medical treatment, or monitored for the development of a disease.
“Multiclass classifier” refers to an algorithm able to solve a classification task using at least two classes.
“Pseudonymized data” refers to personal data that had underwent a processing in such a manner that said personal data can no longer be attributed to a specific subject without using additional information to ensure that the personal data are not attributed to an identified or an identifiable natural person, such as for example by replacing all identifying information in the personal data by an internal identifier stored separately from the personal data itself.

### BRIEF DESCRIPTION OF THE DRAWINGS

FIG. 1 is a work flow providing a schematic representation of the steps of the method invention according to one embodiment of the present.

FIG. 2 is a graph representing the values of the Itakura distance for each training sample for the first training set.

### REFERENCES

CLAS₁—first classification;
CLAS₂—second classification;
CS—step of computing the spectrum;
CSD—step of calculating the spectral distance;
DB—database;
D_{IT}—spectral distance;
EXT—step of extracting at least one feature value from the electroencephalographic signal segment;
F—feature value;
REC—step of receiving at least one segment of electroencephalographic signal;
S—segment of electroencephalographic signal;
Sa—segment of electroencephalographic signal comprising artefacts;
TAG—quality class;
TAG₁—first quality class;
TAG₂—second quality class;
TAG₃—third quality class;
TAG₂_m—muscular artifact;
TAG_N—non-exploitable class;
TAG_N1—first non-exploitable class;
TAG_N2—second non-exploitable class;
Thr—predefined threshold;
TR1—first training set;
TR2—second training set
TS1—training samples;
TS2—second training set;

### DETAILED DESCRIPTION

The following detailed description will be better understood when read in conjunction with the drawings. For the purpose of illustrating, the block diagram comprising the step of the method are shown in the preferred embodiments. It should be understood, however that the application is not limited to the precise arrangements, structures, features, embodiments, and aspect shown. The drawings are not drawn to scale and are not intended to limit the scope of the claims to the embodiments depicted. Accordingly, it should be understood that where features mentioned in the appended claims are followed by reference signs, such signs are included solely for the purpose of enhancing the intelligibility of the claims and are in no way limiting on the scope of the claims.

This invention relates to a method for assessing the quality of an EEG signal using a multiclass classification method. Said method may be implemented as well for any other type of signal, preferably electrophysiological signal recorded from any mammal.

According to one embodiment, the classification method proposed in the present invention is configured to detect the presence of artifacts on each single-channel of an incoming EEG signal.

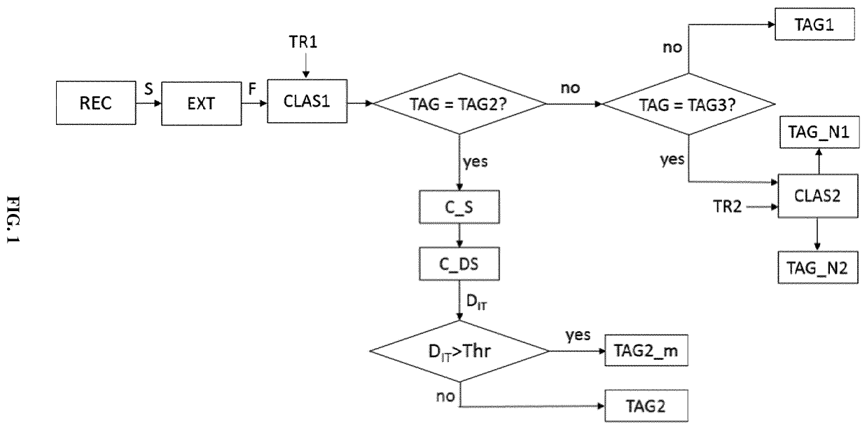

As illustrated in FIG. 1, the method of the present invention comprises a preliminary step of reception REC of at least one segment of electroencephalographic signal S of a subject. Said segment of electroencephalographic signal S may have been acquired from one or multiple channel(s) and as a function of time.

According to one embodiment, the EEG signal segment S received is recorded from one or a plurality of electrodes, positioned onto predetermined areas of the scalp of the subject in order to obtain a one-channel or multi-channel electroencephalographic signal. The electroencephalographic signals may be acquired by at least 1, 2, 4, 8, 10, 15, 16, 17, 18, 19, 20, 21, 32, 64, 128, 256 or more electrodes. In a preferred embodiment, the method receives signals from two or more electrodes. The electrodes may be placed on the scalp according to the 10-10 or 10-20 system, dense-array positioning or any other electrodes positioning known by the man skilled in the art.

The EEG signal segment S received may be obtained with a standard recording module with sampling frequency of at least 200 Hz, notably with sampling frequency of 250 Hz, 500 Hz or 1000 Hz.

The EEG signal segment S may be received in real time or, alternatively, multiples and consecutive EEG signal segment S are recorded during a predefined period of time and stored in a storage medium in order to be analyzed afterwards offline. According to another embodiment, said at least one EEG signal segment S is obtained from a storage medium or a database, such as for example a medical database.

According to one embodiment, the method of the present invention further comprises a pre-processing step consisting in an offset correction of the EEG signal segment S in order to correct for eventual drift over time and/or variation of direct current offsets of one or more EEG channels.

In case of real time implementation of the multiclass classification method of the present invention, the time duration of the electroencephalographic signal S received is adapted. For example, the electroencephalographic signal S received as input may be segmented in consecutive non-overlapping epochs of time duration adapted for real time implementation of the method of the present invention. In one example, such time duration ranges from 0.5 to 2 seconds.

According to one embodiment, the method of the present invention further comprises a pre-processing step consisting in the application of one or more filters to the EEG signal segment S.

The EEG signal segment S from individual scalp electrodes may be digitally filtered with at least one filter chosen from group: low-frequency reject filter, high-frequency reject filter, bandpass filter, band-stop filter or notch filter.

The filtering step may be followed by a down sampling operation or preceded by a down sampling operation.

According to the embodiment represented in FIG. 1, the method further comprises a step consisting in the extraction EXT of at least one feature value F from the electroencephalographic signal segment S. Artifacts and non-EEG signals polluting the EEG signal S are identified via a wide range of different features which characterize their different properties on the base of their time series topology, their spectral template, and/or statistical properties of either univariate or multivariate EEG.

Choosing informative, discriminating and independent features is a crucial step for effective algorithms in classification especially when using the pattern recognition paradigm. The selection of the features to extract in order to reduce the dimension of the feature vector is done before the training of the classifier.

According to one embodiment, the extracted features F comprise at least a time domain feature, a frequency domain feature and/or an entropy feature.

According to one embodiment, the extracted features F are calculated channel by channel of the electroencephalographic signal segment. According to one embodiment, each extracted feature F is extracted from each one of the single-channels. Since each feature can be computed on one single-channel, even if the method is configured to evaluate the quality of EEG signal in multiple individual channels, it will advantageously keep working in the case wherein the signals from all the channels are lost except one.

A time domain feature F could be the rate of zero-crossing of the electroencephalographic signal over a fixed threshold. Said fixed threshold may be the isoelectric line, corresponding to an amplitude of zero. The zero-crossings may be identified as the points wherein the voltage value passes from below the fixed threshold to above the fixed threshold. Alternatively, the zero-crossings may be identified as the points wherein the voltage value passes from above the fixed threshold to below the fixed threshold. The zero-crossings may be as well identified as the points wherein the voltage value passes from the fixed threshold regardless weather from below to above or from above to below. This zero-crossing rate may be computed on different derivative of the EEG segment signal S in particular the 1^{st} and the 2^{nd} derivative of the EEG segment signal S.

The at least one feature F in the time domain is chosen from the following non-exhaustive list of time domain features:
median amplitude value of the EEG segment signal S;
mean amplitude value of the EEG segment signal S;
variance of the amplitude of the EEG segment signal S;
variance of the 1^{st} derivative of the EEG segment signal S;
variance of the 2^{nd} derivative of the EEG segment signal S;
root mean square amplitude of the EEG segment signal S;
difference between the highest and lower amplitude values in the EEG segment signal S;
skewness of the amplitude values of the EEG segment signal S or the EEG segment signal S in a specific EEG frequency band;
kurtosis of the amplitude values of the EEG segment signal S or the EEG segment signal S in a specific EEG frequency band;
standard deviation of the EEG segment signal S in a specific EEG frequency band;
maximum of the EEG segment signal S in a specific EEG frequency band;
integrated EEG segment signal S;
mean absolute amplitude value of the EEG segment signal S;
simple square integral of the EEG segment signal S;
V-order 2 and 3 of the EEG segment signal S;
Log detector of the EEG segment signal S;
average amplitude changes of the EEG segment signal S;
difference absolute standard deviation value of the EEG segment signal S;
number of local maxima and minima of the EEG segment signal S;
2^{nd} and 3^{rd} Hjorth parameters of the EEG segment signal S;
non-linear energy of the EEG segment signal S;
autoregressive modelling errors orders 1 to 9 of the EEG segment signal S

The kurtosis and skewness statistical measures attempt to provide some measures of the distribution of amplitude values (an indication of the signals morphological properties). The mean, the median, the standard deviation, the variance, the maximum, the 2^{nd} and 3^{rd} Hjorth parameters are values that characterize the amplitude of the EEG segment signal S. V-order 2 and 3 values are computation derivate from the variance. The integrated EEG, the log detector, the mean absolute amplitude and the simple square integral are several computations based on the summation of the absolute value of each sample in the EEG segment signal S and in this sense provide other representation of the temporal characteristics of the EEG signal. The root mean square amplitude and the difference between the highest and the lowest values directly reflect the extreme values of the amplitude of the EEG segment signal S. The average amplitude changes between two consecutive data points, the difference absolute standard deviation value and the nonlinear energy of the EEG segment signal S provide information about changes in amplitude through time. The number of local maxima and minima, as the zero-crossing rates can give a complementary information about the variation of the EEG segment signal S. The non-linear energy is a measure of high-frequency content of the EEG segment signal S that is usually used to detect spikes. The autoregressive modelling error is the computation of the error between the EEG segment signal S and the autoregressive model.

Frequency features extract spectral properties of the signal and are originally defined for speech recognition and the assessment of electromyogram quality. The at least one feature F in the frequency domain is chosen from the following non-exhaustive list of frequency domain features:
power of the whole spectrum;
ratio spectrum in an EEG frequency band;
non-normalized power of the spectrum in an EEG frequency band;
logarithmic power of the spectrum in an EEG frequency band;
relative power of the spectrum in an EEG frequency band;
wavelet coefficients of each EEG frequency band;
spectral edge frequency (80%, 90%, 95%) of the total spectrum;
power spectrum momentum of orders 0, 1 and 2;
power spectrum center frequency;
spectral root mean square;
index of spectral deformation;
signal-to-noise spectral ratio;
modified median frequency;
modified mean frequency;
10 cepstral coefficients;
5 frequency-filtered band energies;
5 relative spectral differences.

The power of the whole spectrum, the ratio spectrum, the non-normalized, the logarithmic and the relative power of the spectrum in an EEG frequency band, as well the wavelet coefficients, give complementary representations of the power value in global view or in different EEG frequency bands. The spectral edge frequency is an estimation of the frequency below which p percent (p=80, p=90 or p=95%) of the total power of the EEG segment signal S are located. The power spectrum momentum n is computed by the summation of the power density at each frequency multiplied by this frequency raised to the order n. The power spectrum center frequency is the ratio of spectral moments of order n=1 to order n=0. The spectral root mean square and the index of spectral deformation are also based on some ratios between the power spectrum momentum. The signal-to-noise spectral ratio is the ratio of the power of the spectrum to the power of the noise which is defined as the EEG spectrum upper than 30 Hz. The modified median frequency represents the frequency f for which the total power lower than f is equal to the total power higher than f. The modified mean frequency is the weighted average frequency computed over the amplitude spectrum. The cepstral coefficients are generally used in speech recognition and are computed by applying the discrete cosine transform or the inverse Fourier transform to the logarithmic power of the spectrum of the EEG frequency bands. The frequency-filtered band energies and the relative spectral differences provide information about changes in the different spectrum bands. The frequency-filtered band energy is computed as the subtraction between the logarithmic power spectrum of two EEG frequency bands. A relative spectral difference is a ratio of linear combinations of non-normalized power of the spectrum of several EEG frequency bands.

According to one embodiment, the feature F is calculated in the total spectrum or in different frequency bands, such as for example the delta band (0.5-4 Hz), the theta (4-8 Hz), the alpha band (8-13 Hz), the beta band (13-28 Hz) and the gamma band (28-110 Hz). These frequency bands may be selected using a bandpass filter applied on the desired cutoff frequencies.

In order to obtain the frequency domain features, the length of the EEG signal segment was first artificially increased to the next-higher power of two by adding zero-value samples before transforming the EEG signal segment in its frequency domain with a Fast Fourier Transform.

The at least one feature F may be the spectral entropy feature such as Shannon entropy, spectral entropy or singular value decomposition entropy. The entropy features provide a structural information on the EEG signal segment S.

A complementary measure of quality in the EEG signal segment Qix, also called quality index in the present description, may be computed as a function of the EEG signal S standard deviation. Alternatively, the quality index Qix may be as well function of kurtosis, maximum of absolute value and/or median of absolute values of the EEG signal S.

In one example, the quality index is computed according to the following formula:

$$Qix = \frac{1}{{W}_{j}} {\sum }_{j = 1}^{{N}_{S}} \frac{1}{{e}^{\vert {S}_{j} ( i ) - {T}_{j} \vert \text{/} {S}_{j} ( i )}} - \frac{1}{{W}_{L}} ( {e}^{- max \_ abs ( i )} - {e}^{- med \_ abs ( i )} )$$

Where:
i=Counter indicating the number of EEG segment S;
N_{S}=Number of statistical descriptors;
Wⱼ=[W₁, W₂, . . . W_{NS}]: Weights associated to each statistical descriptor;
Tⱼ=[T₁, T₂, . . . T_{NS}]: Penalizing thresholds;
Sⱼ=[S₁, S₂, . . . S_{NS}]: Type of statistical descriptor;
W_{L}=Weight of the second term.

According to this example, the quality index Qix is calculated using at least one of four different types of statistical descriptors, corresponding to the case wherein N_{S} is equal to 4: kurtosis, standard deviation, maximum of absolute value (max_abs) and median of absolute values (med_abs). The quality index according to the embodiment here above is bounded between 0 (lowest quality) and 1 (highest quality).

According to a preferred embodiment, the feature extraction step comprises the calculation of at least one feature in the time domain, the frequency domain and/or the entropy domain for the EEG signal segment S. The feature extraction step may further comprise the calculation of the quality index Qix. In this embodiment, the extracted features F are arranged in a features vector. The size of the features vector may be reduced to avoid the well-known problem of curse of dimensionality. One of the existing strategies is the Fast Correlation-Based Filter (FCBF) which is a fast subset search algorithm. This feature selection method allows to keep only the features F which are relevant to an EEG signal quality class TAG, measuring the correlation between each feature F and each EEG signal quality class TAG with the symmetrical uncertainty measure (i.e. if two features are redundant, the most relevant one, also called predominant, is selected based on a correlation-based metric). The advantage of using a method implementing symmetrical uncertainty measures is that it is an unbiased measure of predominance. The number of selected features at the end of the procedure depends of a user defined threshold.

According to one embodiment, the quality index Qix is calculated in the feature extraction step and is used as feature for the first classification algorithm.

According to another embodiment, the quality index Qix is used as feature for the second classification algorithm.

According to one embodiment, the electroencephalographic signal segment S is processed using a first classification algorithm. Said first classification algorithm may be a binary classifier or preferably a multiclass classifier.

In one embodiment, said first classifier associates the electroencephalographic signal segment S to at least one class on the basis of a first training set TR1 which comprises multiple training samples TS1. Said training samples TS1 are electroencephalographic signal segment S having known class membership. The class membership of a training samples TS1 may be selected by a visual evaluation of an EEG expert, such as a neurologist. For each of said training samples TS1 of the first training set TR1 is further calculated the at least one feature F chosen during the feature extraction step.

According to one embodiment, the method of the present invention aims to evaluate the quality of EEG signals and the first classifier is configured to associate the EEG signal segment S to an EEG signal quality class TAG. The set of quality classes into which may be classified the EEG signals may be {TAG₁, TAG₂}, {TAG1, TAG₂, TAG₃}, {TAG1, TAG₂, TAG₃, TAG₄} or {TAG₁, TAG₂, . . . TAG_{N}}.

According to one embodiment, the first classification CLAS₁ is performed by a k-nearest neighbors' algorithm. In an alternative embodiment the first classification CLAS₁ is performed by any appropriate classifier-based method as a Support Vector Machine or a Linear or Quadratic Discriminant Analysis. The k-nearest neighbors' algorithm (k-NN) is a non-parametric method used for classification which is widely used for pattern classification. The advantage of using a non-parametric method such the k-NN is that it does not make any assumptions about the probability distribution of the input, therefore no prior knowledge of the data distribution is needed. The k-nearest neighbor algorithm is based on feature similarity. The first classification k-NN assigns to the electroencephalographic signal segment S the quality class which is the most frequent class among the k closest training samples TS1 of the first training set TR1; wherein the distance is calculated between the features values F of electroencephalographic signal segment S and each features values F of the training samples TS1. In other words, the k-NN outputs the class that represents the more probable class based on the k nearest neighbors. The k-NN may output the probability for each class that the EEG signal segment S belongs to one class.

The determination of a neighbor may be performed using many different notions of distance, with the most common being Euclidean and Hamming distance. Euclidean distance is the most popular notion of distance: the length of a straight line between two points Hamming distance between two strings of equal length is the number of positions at which the corresponding symbols are different.

According to one embodiment, the first classification CLAS₁ is performed by a weighted k-nearest neighbors algorithm wherein the contribution of each of the k neighbors is weighted according to their distance to the query point (i.e. unclassified EEG signal segment S), giving for example greater weight wᵢ to closer neighbors or neighbors of a specific class. According to one embodiment, each of the weight wᵢ is calculated with a distance weighting function according to the following formula:

$${w}_{i} = \frac{1}{{d ( {x}_{q} , {x}_{i} )}^{2}}$$

with x_{q}, the feature value F of the EEG signal segment S and xᵢ, the feature value F of one of the k neighbors from TR1.

A further advantage of k-NN is that it implies a type of lazy learning, which is a learning method that generalizes data in the testing phase, rather than during the training phase. This is contrasted with eager learning, which generalizes data in the training phase rather than the testing phase. A benefit of lazy learning is that it can quickly adapt to changes, since it is not expecting a certain generalized dataset.

The optimal choice of k depends upon the data; generally, larger values of k reduces effect of the noise on the classification but make boundaries between classes less distinct. A good k can be selected by various heuristic techniques. According to one embodiment, the choice of k is performed after an analysis of the accuracy of the classification-based method with the variation of the number of k. The optimal k is the k value for which the accuracy of the classification-based method is the best.

According to one example wherein the first classification associates the unclassified EEG signal segment S to one of three quality classes {TAG1, TAG₂, TAG₃}, the training dataset TR1 is composed of a first, a second and a third subset of training samples TS1, each subset comprising training samples labeled with the same quality class.

In this example, the first subset, comprising training samples associated to the first quality class (TAG₁), is characterized by the fact that all training samples acquired in a similar experimental condition wherein the electrodes are placed on the subject in a first predefined contact configuration and wherein the subject is in a first predefined physiological state. The predefined contact configuration of the electrodes being characterized by a predefined number of electrodes displaced in predefined locations on the scalp of the subject (i.e. according to a 10-20 system) and a contact condition between the electrodes and the subject's scalp, depending on the surface of contact and the number of points of contact between the electrodes and the subject's scalp and the pressure exercised by each electrode on the subject's scalp. In this first contact configuration, the contact condition is characterized by a surface of contact of at least the 50% of the sensitive electrode surface, the electrode has at least 2 regions of contact with the subject's scalp and the pressure exercised is superior of a predefined threshold. Said first predefined physiological state is a state of mind and body such as a state of rest or sleep during which the subject may have eyes closed and have relative inhibition of muscles in a predefined area of the body. In order to put the subject in this first predefined physiological state, he/she might be instructed not to move, not to contract facial muscles during signal acquisition and to close the eyes or to avoid to move his/her eyes. Furthermore, the training sample for the first subset may be acquired in a predefined electromagnetic environment characterized by a level of electromagnetic noise inferior to a predefined threshold. Such signal acquisition environment may be obtained in an electromagnetically shielded room to eliminate any electromagnetic environmental contamination of the EEG signal (i.e. cellphone signals and the like). Moreover, the EEG could be preprocessed to reject or correct eventual artefact signals (as powerline noise). Therefore, the first subset, associated to the first quality class (TAG₁), is used as reference for “clean” EEG signals corresponding to an EEG signal free from artifacts and others non-EEG signal.

In this example, the second subset, comprising training samples associated to the second quality class (TAG₂), is characterized by the fact that all training samples are acquired in a similar experimental condition wherein the electrodes are positioned on the scalp in the first predefined contact configuration of the subject and wherein the subject is in a second predefined physiological state. Said second predefined physiological state which generated artifact in the acquired EEG signal is characterized by voluntary contraction of muscles in a predefined region of the subject's body. In order to put the subject in this second predefined physiological state, he/she may be instructed to eye blink, move the head, speak or do any type of movement capable of generating electromyographic signal which would be acquired by the EEG electrodes. Therefore, the second subset of training samples, associated to the second quality class (TAG₂), is used as reference for “artifacted” EEG signals.

In this example, the third subset, comprising training samples associated to the third quality class (TAG₃), is characterized by the fact that all training samples have been acquired in a similar experimental condition wherein the electrodes are place on the subject in a second predefined contact configuration. Said second predefined contact condition wherein the contact surface between the electrode sensitive surface and the subject's scalp is inferior to 50% of the electrode sensitive surface and/or the points of contacts are inferior to two and/or the pressure exerted inferior of a predefined threshold depending on the morphology of the person's head. Therefore, the third subset, associated to the third quality class (TAG₃), is used as reference for “non-exploitable” EEG signals, corresponding to signal acquired from the recording electrodes peels off or electrodes that are moved or the like.

Given this configuration of training dataset TR1, the vector of features F can be reduced so that the three training dataset subsets fill three regions of the features space which are at least partially non-overlapping.

According to one embodiment, the at least one feature F extracted and the k value of the k of the nearest neighbor algorithm are chosen so to have an optimal assessment EEG signal segment S quality. In one embodiment, the feature F are automatically chosen during a computer implemented features extraction step.

According to one embodiment, the first classification step is followed by a misclassification verification step. In order to detect non-exploitable EEG signal segment that might have been misclassified, several parameters may be calculated and confronted to predefined thresholds. According to one example, the percentage of time during which the EEG signal segment S has an approximately constant value may be calculated and may be compared to a predefined threshold. In another example, the amplitude variation may be calculated according to the formula

$$A = \sqrt[2]{2} \sqrt{mean ( {( {A}_{S} - mean - ( {A}_{S} ) )}^{2}} ,$$

wherein Aₛ is the amplitude of the EEG signal segment S and said amplitude variation may be compared to a predefined threshold. For example, if one or both these values exceed the relative predefined thresholds, the EEG signal segment is associate to the quality class of non-exploitable EEG signal (TAG₃).

According to an alternative embodiment, a first and a second set of features are selected and the first classification step CLAS₁ is performed for said first set and said second set of features. In this embodiment, the classification results obtained from the two classifications are compared to obtain a more robust final classification of the EEG signal segment S. The comparison may be a weighted sum of the probability results obtained from the two classifications.

The quality index Qix may be part of one or both sets of features or be used in combination with the first and second classification results, notably to validate the goodness of the classification results.

### Second Classification

According to one embodiment, the method of the present invention comprises a second classification step CLAS₂. Said second classification CLAS₂ concerns the electroencephalographic signal segments S that are classified as “non-exploitable” EEG signal segment in quality class (TAG₃). According to this embodiment, the “non-exploitable” EEG signal segments issued of the first classification are associated to at least one non-exploitable class. The set of non-exploitable classes into which may be classified the non-exploitable EEG signal segments may be {TAG_N₁, TAG_N₂}, {TAG_N₁, TAG_N₂, TAG_N₃} or {TAG_N₁, TAG_N₂, TAG_N_{N}}.

The second classification CLAS₂ may be performed with a classification-based method. According to one embodiment, this classification is done by a weighted k-nearest neighbors' algorithm using a second training set TR2. Said second training set TR2 comprising multiples training samples TS2. Said training samples TS2 are electroencephalographic signal segment S having known class membership. The class membership of a training samples TS2 may be selected by a visual evaluation of an EEG expert, such as a neurologist. For each of said training samples TS2 of the first training set TR2 is further calculated the at least one feature F chosen during the feature extraction step.

According to one embodiment, the second classification CLAS₂ is performed by a weighted k-nearest neighbors' algorithm. According to one embodiment, each of the weight wᵢ for second classification CLAS₂ is calculated with a distance weighting function according to the following formula:

$${w}_{i} = \frac{1}{{d ( {x}_{q} , {x}_{i} )}^{2}}$$

with x_{q}, the feature value F of the non-exploitable EEG signal segment S and xᵢ, the feature value F of one of the k neighbors from TR2.

According to one embodiment, the choice of k is performed after an analysis of the accuracy of the classification-based method with the variation of the number of k. The optimal k is the k value for which the accuracy of the classification-based method is the best.

According to one embodiment wherein the second classification associates the EEG signal segment S classified as TAG₃ to one of two non-exploitable classes {TAG_N₁, TAG_N₂}. The training dataset TR2 is composed of a first and a second subset of training samples TS2, each subsample comprising training samples labeled with the same non-exploitable class. In this example, the first subset, comprising training samples associated to the first non-exploitable class (TAG_N₁), is characterized by the fact that all training samples have been acquired in a similar experimental condition wherein the electrodes are place on the subject in the second predefined contact configuration. Therefore, this first subset, associated to the first non-exploitable class (TAG_N₁), is again used as reference for “non-exploitable” EEG signals corresponding to signal acquired from recording electrodes peels off or electrodes that are moved or the like.

In this example, the second subset, comprising training samples associated to the second non-exploitable class (TAG_N₂), is characterized by the fact that all training samples have been acquired in a similar experimental condition wherein the contact between the electrodes and the scalp of the subject is not ensured anymore. Therefore, the second subset, associated to the second non-exploitable class (TAG_N₂), is used as reference for “non” EEG signals, corresponding to those signals not originating from brain electrical activity but from other sources such as the EEG recording apparatus itself or electronical equipment in the environment.

Given this configuration of training dataset TR2, the vector of features F can be reduced so that the two training dataset subsets fill two regions of the features space which are at least partially non-overlapping. In one embodiment, the feature F are automatically chosen during a computer implemented features extraction step.

According to one embodiment, the choice of k is performed after an analysis of the accuracy of the classification-based method with the variation of the number of k. The optimal k is the k value for which the accuracy of the classification-based method is the best.

According to one embodiment, the at least one feature F extracted and the k value of the k of the second nearest neighbor algorithm are chosen so to have an optimal assessment of the non-exploitable EEG signal segment S.

According to an alternative embodiment, a first and a second set of features are selected and the second classification step CLAS₂ is performed for said first set and said second set of features. In this embodiment, the classification results obtained from the two classifications are compared to obtain a more robust final classification of the EEG signal segment S.

### Muscular Artifact Discrimination

According to one embodiment, the method of the present invention further comprises a succession of steps providing as output an information concerning the source generating the artifact that have been detected by the first classification. In one embodiment, those steps allow the discrimination of muscular artifacts from other source artifacts in electroencephalographic signal.

According to one embodiment, a first step consists in the calculation CS of the spectrum (Pxx1) by Fast Fourier transform in a predefined frequency range for each the EEG signal segment S classified in the quality class (TAG₂). The predefined frequency range f_{R} may be any sub-range comprised in the range [1, 60] Hz.

According to one embodiment, a second step consists in the calculation CSD of a spectral distance D_{IT} between the spectrum (Pxx1) and a reference spectrum (Pxx2). Said reference spectrum (Pxx2) may be computed as the average value of the spectra of at least two electroencephalographic signal segments S in the first training set associated to the first quality class (TAG₁) of clean EEG signals. The spectra of at least two electroencephalographic signal segments S are calculated by Fast Fourier transform in the predefined frequency range f_{R}.

According to one embodiment, the spectral distance D_{IT} is an Itakura spectral distance according to the following formula:

$${D}_{IT} = log ( mean ( \frac{Pxx 2}{Pxx 1} ) ) - mean ( log ( \frac{Pxx 2}{Pxx 1} ) )$$

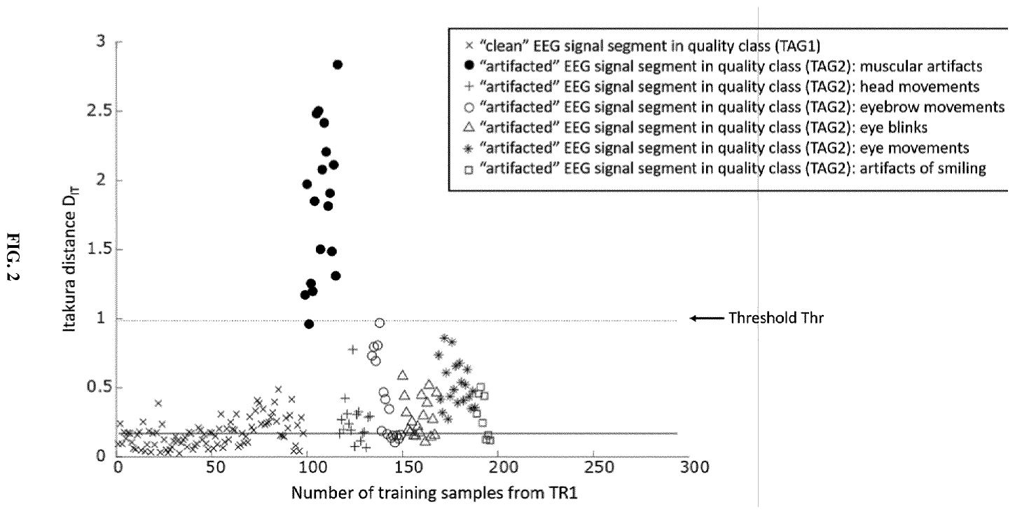

According to one embodiment, a third step consists in the comparison of said spectral distance D_{IT} to a predefined threshold Thr to determine if the artifacted EEG signal segment S comprises a muscular artifact, as shown in FIG. 2. Said predefined threshold may be established by at least one EEG expert. The artifacted EEG signal segment S comprising a muscular artifact may be associate to an artefact class (TAG2_m).

According to one embodiment, the method of the present invention is a computer-implemented method.

The present invention relates to a multiclass classification of an electroencephalographic signal (EEG), comprising the steps of:
determination of the EEG quality of at least one electroencephalographic signal segment associating the electroencephalographic signal segment to a quality class with the method according to any one of embodiment hereabove; and
among the electroencephalographic signal segment identified as comprising artifacts, identification of electroencephalographic signal segment comprising muscular artifacts with the method according to any one of embodiment hereabove.

According to one embodiment, the methods of the present invention are automated computer implemented methods.

The present invention further relates to a method for updating a medical database DB. According to one embodiment, this method comprises a first step of reception of a first set of pseudonymized data concerning a first subject. Said first set of pseudonymized data may comprise at least one electroencephalographic signal segment S and a class to which said electroencephalographic signal segment S has been previously associated by the method for multiclass classification according to any one of the embodiments described hereabove. According to one embodiment, this method further comprises a second step consisting in the update of said database DB by storing the first set of pseudonymized data concerning the first subject. For example, the decision to update the database DB including said pseudonymized data may be based on a comparison of the probability associated to such a quality class with a predefined threshold. This updating method may be implemented for a second subject, a third subject and so on. This updating method may be independent from any subject or may be subject-specific or dependent from another specificity.

The present invention further relates to a computer program for multiclass classification of an electroencephalographic signal, the computer program product comprising instructions which, when the program is executed by a computer, cause the computer to carry out the steps of the computer-implemented method for modifying nociception according to anyone of the embodiments described hereabove in relation.

The invention also relates to a system for the processing of electroencephalographic signals comprising a data processing system comprising means for carrying out the steps of the method according to any one of the embodiment described hereabove.

According to one embodiment, the system further comprises an acquisition set-up for acquiring at least a segment of electroencephalographic signals from a subject. According to one embodiment, the acquisition set-up comprises any means known by one skilled in the art enabling acquisition (i.e. capture, record and/or transmission) of electroencephalographic signals as defined in the present invention, preferably electrodes or headset as explained hereabove. According to one embodiment, the acquisition set-up comprises an amplifier unit for magnifying and/or converting the electroencephalographic signals from analog to digital format.

According to one embodiment, the system comprises an output apparatus to output a visual or auditory stimulus related to the result of the classification.

According to one embodiment, the data processing system is a dedicated circuitry or a general purpose computer device, configured for receiving the data and executing the operations described in the embodiment described above. Said computer device may comprise a processor and a computer program. The data processing system may include, for example, one or more servers, motherboards, processing nodes, personal computers (portable or not), personal digital assistants, smartphones, smartwatches, smartbands, cell or mobile phones, other mobile devices having at least a processor and a memory, and/or other device(s) providing one or more processors controlled at least in part by instructions.

The processor receives digitalized neural signals and processes the digitalized electroencephalographic signals under the instructions of the computer program to classify the signal. According to one embodiment, the computing device comprises a network connection enabling remote implementation of the method according to the present invention. According to one embodiment, electroencephalographic signals wirelessly communicated to the data processing system. According to one embodiment, the output generator wirelessly receives the classes associated to the electroencephalographic signal segments S from the data processing device.

The present invention further relates to a non-transitory computer-readable storage medium comprising instructions which, when the computer program is executed by a data processing system, cause the data processing system to carry out the steps of the method according to anyone of the embodiments described hereabove.

Computer programs implementing the method of the present embodiments can commonly be distributed to users on a distribution computer-readable storage medium such as, but not limited to, an SD card, an external storage device, a microchip, a flash memory device, a portable hard drive and software websites. From the distribution medium, the computer programs can be copied to a hard disk or a similar intermediate storage medium. The computer programs can be run by loading the computer instructions either from their distribution medium or their intermediate storage medium into the execution memory of the computer, configuring the computer to act in accordance with the method of this invention. All these operations are well-known to those skilled in the art of computer systems.

While various embodiments have been described and illustrated, the detailed description is not to be construed as being limited hereto. Various modifications can be made to the embodiments by those skilled in the art without departing from the true spirit and scope of the disclosure as defined by the claims.

1-18. (canceled)

19. A method for assessing the quality of an electroencephalographic signal based on a multiclass classification, wherein said method comprises:
receiving at least one segment of electroencephalographic signal acquired from at least one electrode;
extracting at least one feature value from each channel of the electroencephalographic signal segment;
classifying with a first classification so as to assign each channel of the electroencephalographic signal segment to one of at least three quality classes: {TAG₁, TAG₂, . . . , TAG_{N}};
wherein said first classification is performed by a k-nearest neighbors' algorithm:
using a first training set comprising multiples training samples, wherein each training sample of the first training set is associated to one of the quality classes and to at least one feature value;
assigning to each channel of the electroencephalographic signal segment the quality class which is the most frequent class among the k training samples of the first training set which are nearer to each channel of the electroencephalographic signal segment; wherein the distance is calculated between the feature value of each channel of the electroencephalographic signal segment and each feature value of the training samples; and
outputting said quality class for each channel of the electroencephalographic signal segment.

20. The method according to claim 19, wherein the at least one feature and the k value of the k-nearest neighbors' algorithm are configured so that:
the first quality class is associated to EEG signal segment acquired with the electrodes positioned according to a first predefined configuration of contact between the electrodes and a subject' scalp and during a first predefined physiological state of a subject; and/or
the second quality class is associated to EEG signal segments acquired with the electrodes positioned according to a first predefined configuration of contact between the electrodes and a subject' scalp and during a second physiological state of a subject; and/or
the third quality class corresponds to EEG signal segments acquired with the electrodes positioned according to a second predefined configuration of contact between the electrodes and a subject' scalp.

21. The method according to claim 19, wherein the feature is a quality index, function of the standard deviation of the electroencephalographic signal segment.

22. The method according to claim 21, wherein the quality index is further function of kurtosis, maximum of absolute value and/or median of absolute values.

23. The method according to claim 19, wherein the at least one feature of the electroencephalographic signal segment is chosen from the following list of features:
the rate of zero-crossings of the electroencephalographic signal segment over a fixed threshold;
power spectrum moments of different orders;
index of spectral deformation;
modified median frequency.

24. The method according to claim 19, wherein the first classification is performed by a weighted k-nearest neighbors' algorithm.

25. The method according to claim 19, wherein the segment of electroencephalographic signal is acquired from at least two electrodes.

26. The method according to claim 19, further comprising a second classification assigning the electroencephalographic signal segment classified in quality class to one of at least two non-exploitable classes: {TAG_N₁, TAG_N₂, . . . , TAG_N_{N}}, wherein the electroencephalographic signal segment classified in the non-exploitable classes are the electroencephalographic signal segments excluded from further analysis.

27. The method according to claim 26, wherein the second classification is performed with a weighted k-nearest neighbors' algorithm using a second training set, said second training set comprising multiples training samples, wherein each training sample of the second training set is associated to one of the non-exploitable classes and to at least one feature value.

28. The method according to claim 27, wherein the at least one feature and a k value of the k-nearest neighbors' algorithm of the second classification are configured so that:
the first non-exploitable class is associated to EEG signal segment acquired with the electrodes positioned according a second predefined configuration of contact between the electrodes and a subject' scalp; and
the second non-exploitable class is associated to EEG signal segment acquired with electrodes having no physical contact with a subject's scalp.

29. The method according to claim 19, further comprising discrimination of muscular artifacts from other source artifacts in electroencephalographic signal by:
for each EEG signal segment classified in the quality class computing the spectrum by Fourier transform in a predefined frequency range;
calculating of a spectral distance between the spectrum of each EEG signal segment and a reference spectrum; and
comparing said spectral distance to a predefined threshold to determine the presence of a muscular artifact in the EEG signal segment in the quality class and assign it a class.

30. The method according to claim 29, wherein the reference spectrum is computed as the average value of the spectra of at least two electroencephalographic signal segments, wherein said at least two electroencephalographic signal segments are acquired with the electrodes positioned according to a first predefined configuration of contact between the electrodes and a subject' scalp and during a first predefined physiological state of a subject.

31. The method according to claim 29, wherein the spectral distance is an Itakura spectral distance.

32. A method for updating a database, said method comprising:
receiving a first set of pseudonymized data concerning a first subject; wherein said first set of pseudonymized data comprises at least one segment of electroencephalographic signal segment and a class to which said segment of electroencephalographic signal has been previously associated by the method according to claim 19; and
updating said first database by storing the first set of pseudonymized data concerning the first subject.

33. A system for assessing the quality of an electroencephalographic signal (EEG) based on a multiclass classification, said system comprising a data processing system having:
at least one input adapted to receive at least one segment of electroencephalographic signal acquired from at least one electrode;
at least one processor configured to iteratively:
extracting at least one feature value from each channel of the electroencephalographic signal segment;
classifying with a first classification so as to assign each channel of the electroencephalographic signal segment to one of at least three quality classes: {TAG₁, TAG₂, . . . , TAG_{N}};
wherein said first classification is performed by a k-nearest neighbors' algorithm:
using a first training set comprising multiples training samples, wherein each training sample of the first training set is associated to one of the quality classes and to at least one feature value; and
assigning to each channel of the electroencephalographic signal segment the quality class which is the most frequent class among the k training samples of the first training set which are nearer to each channel of the electroencephalographic signal segment; wherein the distance is calculated between the feature value of each channel of the electroencephalographic signal segment and each feature value of the training samples;
at least one output adapted to provide said quality class for each channel of the electroencephalographic signal segment.

34. The system of claim 33, further comprising an acquisition set-up for acquiring at least one segment of electroencephalographic signals from a subject.

35. The system according to claim 33, wherein the at least one feature and the k value of the k-nearest neighbors' algorithm are configured so that:
the first quality class is associated to EEG signal segment acquired with the electrodes positioned according to a first predefined configuration of contact between the electrodes and a subject' scalp and during a first predefined physiological state of a subject; and/or
the second quality class is associated to EEG signal segments acquired with the electrodes positioned according to a first predefined configuration of contact between the electrodes and a subject' scalp and during a second physiological state of a subject; and/or
the third quality class corresponds to EEG signal segments acquired with the electrodes positioned according to a second predefined configuration of contact between the electrodes and a subject' scalp.

36. The system according to claim 33, wherein the at least one processor is further configured to comprise a second classification assigning the electroencephalographic signal segment classified in quality class to one of at least two non-exploitable classes: {TAG_N₁, TAG_N₂, . . . , TAG_N_{N}}, wherein the electroencephalographic signal segment classified in the non-exploitable classes are the electroencephalographic signal segments excluded from further analysis.

37. The system according to claim 33, wherein the at least one processor is further configured to discriminate muscular artifacts from other source artifacts in electroencephalographic signal by:
for each EEG signal segment classified in the quality class computing the spectrum by Fourier transform in a predefined frequency range;
calculating of a spectral distance between the spectrum of each EEG signal segment and a reference spectrum; and
comparing said spectral distance to a predefined threshold to determine the presence of a muscular artifact in the EEG signal segment in the quality class and assign it a class.

38. A non-transitory computer-readable storage medium comprising instructions which, when the program is executed by a computer, cause the computer to carry out the method according to claim 19.
